(19) United States
(12) Patent Application Publication
(10) Pub. No.: US 2026/0130790 A1
(43) Pub. Date: May 14, 2026

(54) DEVICES AND METHODS FOR TREATMENT OF SKIN CONDITIONS

(71) Applicants: Mitt Coats, Providence, RI (US); Carlos Wambier, Hamden, CT (US)

(72) Inventors: Mitt Coats, Providence, RI (US); Carlos Wambier, Hamden, CT (US)

(21) Appl. No.: 19/338,437

(22) Filed: Sep. 24, 2025

Related U.S. Application Data
(60) Provisional application No. 63/698,148, filed on Sep. 24, 2024.

(51) Int. Cl. A61F 7/02; A61F 7/00; A61F 13/00; A61F 13/05

(52) U.S. Cl. CPC A61F 7/02; A61F 13/00063; A61F 13/05; A61F 2007/0093; A61F 2007/0219; A61F 2007/0226; A61F 2007/0263; A61F 2007/0282; A61F 2013/00906; A61F 2013/00961

(57) ABSTRACT

Provided herein are devices and methods for the treatment of skin conditions. A skin treatment apparatus according to the present disclosure generally includes a treatment head defining a treatment space, the treatment head having a body forming said treatment space. The body is further configured to contact the treatment space to a treatment surface containing a lesion. A treatment media is disposed within the treatment space. The skin treatment apparatus also includes a heating element configured to heat the treatment media.

### CROSS REFERENCE TO RELATED APPLICATIONS

This application depends from and claims priority to U.S. Provisional Application No.: 63/698,148 filed Sep. 24, 2024, the entire contents of which are incorporated herein by reference.

### TECHNICAL FIELD

Embodiments of the present disclosure are generally related to devices and methods for the treatment of skin conditions.

### BACKGROUND

Warts are caused by infection with the human papillomavirus (HPV). These skin growths can lead to significant discomfort, pain, and disruption of daily life. It is estimated that approximately 10% of the population suffers from warts at any given time. Up to 33% of these cases become recalcitrant, leading to persistent and significant morbidity.

Existing treatments of recalcitrant warts often cause significant pain, impair mobility, may cause scarring and hyper/hypopigmentation, can be expensive, and often demonstrate limited efficacy. Moreover, there is no cure or universally accepted standard of care for the treatment of recalcitrant warts.

Accordingly, there is a need to develop treatment options that offer higher efficacy with fewer side effects and lower cost.

### SUMMARY

Embodiments of the present disclosure are directed to devices and methods for the hydrohyperthermic treatment of skin conditions.

In an embodiment, the present disclosure relates to a skin treatment apparatus comprising a treatment head defining a treatment space, the treatment head comprising a body forming said treatment space, wherein said body is further configured to contact the treatment space to a treatment surface comprising a lesion; a treatment media disposed within the treatment space; and a heating element configured to heat the treatment media. Optionally, the treatment media is a fluid or a gel. Optionally, the treatment media includes one or more therapeutic agents. In some embodiments, the heating element is configured to heat the treatment media to a predetermined temperature, wherein the predetermined temperature is from about 40° C. to about 100° C. Optionally, the skin treatment apparatus comprises a temperature sensor contacting the heating element and/or the treatment media. In some embodiments, the skin treatment apparatus includes a pressure sensor In some embodiments, the skin treatment apparatus includes an impedance sensor. In some embodiments, the skin treatment apparatus includes a display interface configured to display one or more parameters selected from treatment protocol, treatment duration remaining, treatment duration elapsed, temperature of the treatment media, or error messages. Optionally, the skin treatment apparatus is configured to send or receive data about a treatment to an external device. In some embodiments, the treatment head seals to the treatment surface adjacent to the lesion via adhesion. In some embodiments, the treatment head seals to the treatment surface adjacent to the lesion via suction. In some embodiments, the skin treatment apparatus further comprises a controller communicatively coupled to the heating element, the controller configured to activate the heating element during a treatment protocol.

In another embodiment, the present disclosure relates to a method of treating a lesion using the skin treatment apparatus, the method comprising: sealing the treatment head to a treatment surface comprising the lesion; initiating a treatment protocol, the treatment protocol having a treatment duration; heating the treatment media to a predetermined temperature; and contacting the lesion with the heated treatment media for the treatment duration. Optionally, the lesion is selected from warts, bacterial infections, viral infections, insect bites, acrochordons, skin cancer, corns, calluses, actinic keratosis, molluscum contgaiosum, dermatosis papulosa nigra, solar lentigines, cutaneous horns, lichen planus, keloids, pyogenic granulomas, or granuloma annulare. Optionally, the lesion is a wart. In some embodiments, the treatment duration is from about 10 seconds to about 60 seconds. In some embodiments, the treatment duration is from about 1 minute to about 60 minutes. In some embodiments, the predetermined temperature is from about 40° C. to about 100° C. In some embodiments, sealing the treatment head to a treatment surface comprises activating a vacuum pump to remove air from the treatment space. In some embodiments, the treatment protocol further comprises a plurality of cycles, wherein each cycle comprises: heating the treatment media to a first predetermined temperature and maintaining the treatment media at the first predetermined temperature for a first treatment time; deactivating said heating element for a second treatment time.

Additional features and advantages of the embodiments described herein will be set forth in the detailed description which follows, and in part will be readily apparent to those skilled in the art from that description or recognized by practicing the embodiments described herein, including the detailed description which follows, and the claims.

### BRIEF DESCRIPTION OF THE DRAWINGS

FIG. 1 depicts a schematic cross-sectional view of an exemplary skin treatment apparatus, according to one or more embodiments shown and described herein;

FIG. 2 schematically depicts an exemplary skin treatment apparatus, according to one or more embodiments shown and described herein;

FIG. 3 depicts a schematic of a system for controlling a skin treatment apparatus, according to one or more embodiments shown and described herein;

FIGS. 4A-C depict a subject undergoing treatment according to one or more embodiments described herein. FIG. 4A depicts the lesion prior to treatment. FIG. 4B depicts the lesion after thirteen months of conventional treatments. FIG. 4C depicts resolution of the lesions after treatment according to one or more embodiments described herein.

### DETAILED DESCRIPTION

Embodiments of the present disclosure are directed to skin treatment apparatuses and associated methods of use.

The disclosure should not be construed as limited to the embodiments set forth herein. Rather, these embodiments are provided so that this disclosure will be thorough and complete, and will fully convey the subject matter to those skilled in the art.

### Definitions

Unless otherwise defined, all technical and scientific terms used herein have the same meaning as commonly understood by one of ordinary skill in the art. The terminology used in the disclosure herein is for describing particular embodiments only and is not intended to be limiting.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used in the specification and the appended claims, the singular forms “a,” “an,” and “the” are intended to include the plural forms as well, unless the context clearly indicates otherwise. Thus, for example, reference to “a” component includes aspects having two or more such components, unless the context clearly indicates otherwise.

Embodiments of the present disclosure are generally directed to devices and methods for the treatment of the skin and other near surface tissues, such as the dermis and/or hypodermis, in a subject. In some embodiments, the devices and methods described herein are for the treatment of one or more skin conditions in a subject.

As used herein, “subject” and grammatical equivalents thereof refers to a human or animal subject suffering from a skin condition. In some embodiments, the skin condition may be treated with one or more embodiments of the present disclosure.

A “skin condition” as provided herein may be any skin condition associated with one or more lesions on the skin or near-surface tissues. Exemplary non-limiting skin conditions include warts (including verruca vulgaris, verruca plantaris, verruca plana, filiform warts, periungual warts, mosaic warts, genital warts, butcher's warts, etc.) acrochordons, acne, skin cancer (e.g., basal cell carcinoma, squamous cell carcinoma, verrucous carcinoma), corns, calluses, actinic keratosis, molluscum contgaiosum, dermatosis papulosa nigra, solar lentigines, cutaneous horns, lichen planus, keloids, pyogenic granulomas, granuloma annulare, and the like. In some embodiments, the skin condition is a lesion, optionally selected from lesion is selected from warts, bacterial infections, viral infections, insect bites, acrochordons, skin cancer, corns, calluses, actinic keratosis, molluscum contgaiosum, dermatosis papulosa nigra, solar lentigines, cutaneous horns, lichen planus, keloids, pyogenic granulomas, or granuloma annulare. In some embodiments, the skin condition is warts. In some embodiments, the skin condition is recalcitrant wart(s).

A “recalcitrant wart” is a wart that is refractory to standard treatment. Refractory to standard treatment is failure to effectively treat the wort with two different treatments or after three or more months of a single conventional treatment.

Devices disclosed herein generally include a skin treatment apparatus that includes a treatment head defining a treatment space, the treatment head comprising a body forming the treatment space, wherein the body is further configured to contact the treatment space to a treatment surface comprising a lesion, wherein the apparatus further includes a treatment media disposed within the treatment space, and a heating element configured to heat the treatment media.

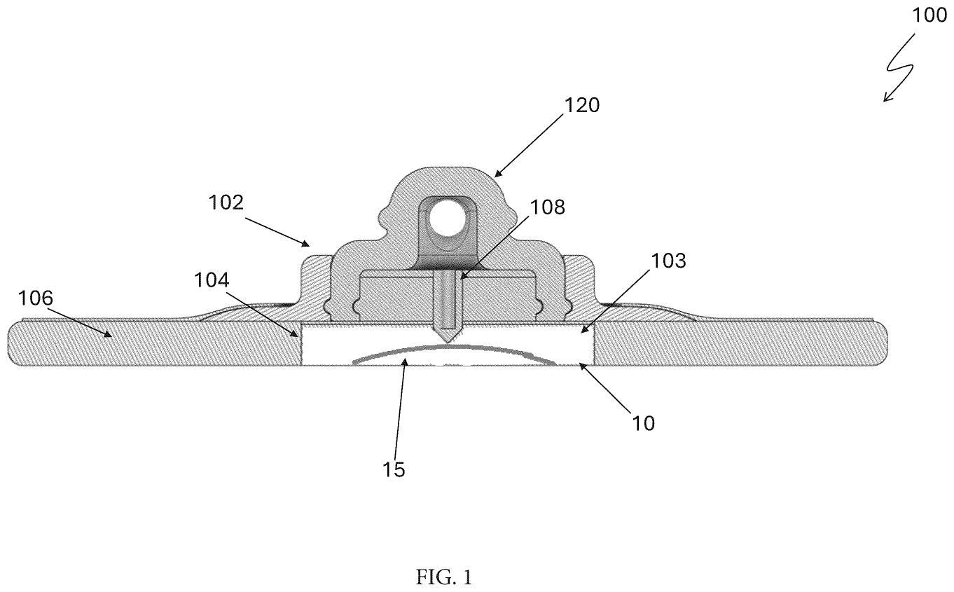

Referring to the drawings, FIG. 1 depicts a skin treatment apparatus 100 according to one or more embodiments as provided herein. In some embodiments, the skin treatment apparatus 100 includes a treatment head 102.

In some embodiments, the treatment head 102 is configured to be removably coupled to the skin treatment apparatus 100. For example, and without being bound by theory, in some embodiments, such as shown in FIG. 1, the treatment head 102 is removably coupled to the treatment apparatus 100 via a gasket. Other suitable coupling mechanisms, such as O-rings, silicone seals, mechanical friction, and the like, are contemplated and possible. Optionally, a treatment head 102 is integrated into the skin treatment apparatus 100 such that the treatment head is not removable from the remainder of the skin treatment apparatus 100.

A skin treatment apparatus 100 may include a plurality of treatment heads 102. Optionally, each treatment head 102 of the plurality of treatment heads is adapted to accommodate the needs of the subject and/or the treatment. For example, and without being bound by theory, in some embodiments, the plurality of treatment heads may have a variety of sizes to effectively treat different lesions. In some embodiments, the plurality of treatment heads may have a variety of shapes to effectively treat lesions in different anatomical positions. In some embodiments, the plurality of treatment heads may be formed from a variety of materials to better maintain therapeutic parameters for treating lesions caused by different skin conditions.

Optionally, the treatment head 102 defines a treatment space 103. Any suitable shape and/or size of the treatment head 102 is contemplated and possible. In some embodiments, the treatment head 102 is concave, defining a treatment space 103 for treating a lesion, described in greater detail herein. In some embodiments, the treatment space 103 is defined by interior boundaries of the treatment head 102 and is configured to isolate and target the lesion. In some embodiments, various therapeutic parameters, such as temperature, pressure, and/or fluid flow, are controlled within the treatment space 103 to target a specific lesion, described in greater detail herein. For example, and without being bound by theory, heated fluid and/or energy (e.g., infrared, microwave) may be delivered to the treatment space 103 and be maintained within target parameters within the space, directly impacting the lesion 15 when the skin treatment apparatus is applied to a treatment surface. It will be appreciated that this targeting of the lesion 15 concentrates the therapy at the lesion 15 minimizing the impact on surrounding tissue, thereby reducing the risk of side effect(s).

It will be appreciated that the size and shape of the treatment head 102 and/or the treatment space 103 may be adapted based on specific needs of a subject. In some embodiments, the treatment space 103 is insulated and/or sealed by the treatment head 102. Optionally, and described in greater detail herein, the treatment head 102 is adapted to maintain consistent therapeutic conditions in the treatment space 103 for the duration of treatment. For example, and without being bound by theory, in some embodiments, the treatment head 102 includes one or more sensors, described in greater detail herein. It will be appreciated that the sensor(s) enables the treatment space 103 to be substantially maintained at the desired temperature, pressure, and/or fluid levels, to effectively treat the lesion 15.

In other embodiments, the treatment head is configured to permit controlled alteration of one or more therapeutic conditions in the treatment space 103 for the entire or a portion of the duration of treatment. For example, and without being bound by theory, in some embodiments, the treatment head 102 includes one or more sensors, described in greater detail herein. It will be appreciated that the sensor(s) enables measurement of one or more therapeutic conditions in the treatment space 103 and to allow the skin treatment apparatus to alter the treatment condition for any portion of the treatment time such that the treatment condition for a portion of the treatment may be applied at the desired temperature, pressure, and/or fluid levels, to effectively treat the lesion 15.

In some embodiments, the treatment space 103 facilitates the circulation and/or application of the treatment media to the lesion 15. As described in greater detail herein, the treatment media may be any substance that adequately treats the lesion, such as fluids, hydrogels, gels, ointments, pastes, creams, combinations thereof, and the like.

In some embodiments, the treatment head 102 includes a body 104. Optionally, the body 104 is hollow or semi-hollow, defining the boundaries of the treatment space 103. In some embodiments, the body 104 is configured to maintain the therapeutic conditions for treating the lesion. In some embodiments, the body 104 enables fluid communication between the treatment space 103 and other components of the skin treatment apparatus 100, described in greater detail herein. Optionally, the body 104 may define one or more channels or conduits 105 to allow for the circulation of heated fluid and/or the application of energy to the treatment space 103.

Any suitable material for forming the treatment head 102 and/or body 104 is contemplated and possible. In some embodiments, the treatment head 102 and/or body 104 is formed from a material that conforms to the contours of the skin. In some embodiments, the material insulates the treatment space 103. In some embodiments, the treatment head 102 and/or body 104 is formed from a material that prevents interference of external factors, such as ambient temperature, with the therapeutic conditions of the treatments. In some embodiments, the body is formed of a heat-resistant material. Exemplary materials include, but are not limited to silicone, rubber, polytetrafluoroethylene, polyetheretherketone, polyurethane, fluoroelastomers, high-density polyethylene, polypropylene, ethylene propylene diene monomer, combinations thereof, and the like. In some embodiments, the treatment head 102 and/or the body 104 is at least partially transparent, allowing visual alignment over the lesion 15. It will be appreciated that a transparent treatment head 102 enables additional therapies, such as light therapy, to treat the lesion.

The treatment head 102 may be configured to attach to the treatment space 103 via any suitable mechanism, including, but not limited to adhesion, suction, and the like.

In some embodiments, the treatment head 102 includes a lip 106. Optionally, the lip 106 is disposed at a distal end of the skin treatment apparatus 100. In some embodiments, the lip 106 is configured to seal the treatment head 102 to a treatment surface 10, thereby enclosing the treatment space 103. As used herein, “treatment surface” refers to the specific area of the skin that is targeted for therapeutic intervention using the skin treatment apparatus. In some embodiments, the treatment surface 10 refers to the area of skin where the treatment head 102 is applied. Generally, the treatment surface 10 includes the lesion 15 and any adjacent skin necessary to ensure effective treatment of the lesion 15.

Optionally, the lip 106 connects the treatment head 102 to the treatment surface 10. In some embodiments, the lip 106 forms an airtight and/or watertight seal between the skin treatment apparatus 100 and the treatment surface 10. It will be appreciated that the treatment surface 10 further defines at least one external boundary of the treatment space 103 during treatment of the lesion. In some embodiments, the lip 106 maintains the therapeutic parameters in the treatment space 103, described in greater detail herein.

In some embodiments, the lip 106 seals with the treatment surface 10, thereby preventing heat and/or treatment media from escaping. For example, and without being bound by theory, in some embodiments, the lip 106 is configured to compress slightly when pressed against the treatment surface 10, thereby sealing the treatment head 102 to the treatment surface 10. In some embodiments, the lip 106 includes a slightly raised or beveled edge to enhance the seal. In some embodiments, the lip 106 seals to the treatment surface 10 via adhesion. In some embodiments, the lip 106 seals to the treatment surface 10 via suction. In some embodiments, the skin treatment apparatus 100 is further sealed to the skin via a vacuum pump or other suitable means. Optionally, the lip 106 seals to the treatment surface via an adhesive. An adhesive is any art recognized adhesive material suitable for application to the skin of a subject. Illustrative examples of an adhesive include silicones, acrylics, hydrocolloids, or synthetic or natural rubber based adhesives as are used in the art. A treatment apparatus may include one or more adhesives applied to the lip prior to application to the treatment space.

The lip 106 may be formed from any suitable material. In some embodiments, the lip 106 is formed from the same material as the body 104. In some embodiments, the lip 106 is formed of a material with suitable shape and/or flexibility so as to be capable of conforming to natural contours of the treatment surface such as the skin. In some embodiments, the lip 106 is adapted to accommodate motion from the subject and/or variations in skin texture. In some embodiments, the lip 106 is formed from a soft, flexible material. Optionally, the lip 106 is formed from a material that is resistant to heat and wear. In some embodiments, the material is biocompatible and capable of forming a seal without causing discomfort. In some embodiments, the material is hypoallergenic. Exemplary materials used in the formation of a lip include, but are not limited to silicone rubber, thermoplastic elastomers, latex rubbers, polyurethane, ethylene vinyl acetate, polyvinyl chloride, combinations thereof, and the like. In some embodiments, the lip 106 is formed from a silicone material, optionally, a medical-grade silicone. In some embodiments, the lip 106 is formed from or includes a skin adhesive and fabric layer, such as a medical grade tape, though any suitable layer is contemplated and possible.

In some embodiments, the lip 106 is coupled to the body 104. Any suitable coupling mechanism is contemplated and possible, including, but not limited to, adhesive bonding, gaskets, snap-fit connections, threaded connections, magnetic clasps, mechanical interlocking, welding, heat sealing, and the like. In some embodiments, the treatment head 102 is formed as a single unit, wherein the lip 106 is integrated with the body 104.

In some embodiments, the lip 106 is removably coupled to the body 104. For example, and without being bound by theory, in some embodiments, the lip 106 and/or the body 104 may include alignment features such as grooves, ridges, and/or pins that guide the lip 106 into the correct position. Optionally, the lip 106 is interchangeable. In some embodiments, the skin treatment apparatus 100 includes a plurality of lips adapted to form a seal with a variety of treatment surfaces and/or anatomical regions. For example, and without being bound by theory, each lip of the plurality of lips may be adapted to seal a different type of treatment surface, such as smooth flat surfaces, irregular or textured surfaces, surfaces that have greater movement (such as joints) and/or surfaces with hair.

Optionally, the body 104 and/or the lip 106 may be further treated with a coating, such as by spraying, dipping, or painting the coating to the lip 106. Exemplary, non-limiting coating materials include silicone-based coatings, medical-grade adhesive coatings, hydrocolloid coatings, polyurethane coatings, pressure-sensitive adhesive coatings, acrylic adhesive coatings, combinations thereof, and the like.

In some embodiments, the treatment head 102 is a removable cartridge filled with a treatment media, described in greater detail herein. It will be appreciated that use of a treatment media is optional and the energy may be transferred directly to the lesion, described in greater detail herein.

During treatment of the lesion, described in greater detail herein, the treatment head 102 may be attached to the skin treatment apparatus 100. Optionally, the act of attaching the treatment head 102 may pierce a membrane, thereby allowing the treatment media to contact the treatment surface. It will be appreciated that, after treatment, the treatment head 102 may be detached and discarded.

In some embodiments, and still referring to FIG. 1, the treatment space 103 is configured to hold a treatment media. Exemplary, non-limiting treatment media may include water, saline, gels, hydrogels, or any other suitable fluid. In some embodiments, the treatment media is a hydrogel. Optionally, the hydrogel remains viscous at the treatment temperature, described in greater detail herein. In some embodiments, the hydrogel remains viscous at temperature from about 30° C. to about 100° C., though other temperatures and ranges are contemplated and possible.

Exemplary hydrogels include, but are not limited to: polyethylene glycol hydrogels, polyacrylamide hydrogels, polyvinyl hydrogels, poly-N-isopropylacrylamide hydrogels, poly(2-hydroxyethyl methacrylate hydrogels, alginate hydrogels, chitosan hydrogels, polyurethane hydrogels, polyacrylic acid hydrogels, silk fibroin hydrogels, combinations thereof, and the like, though other suitable hydrogel are contemplated and possible.

In some embodiments, the treatment media may contain one or more therapeutic agents. Illustrative nonlimiting examples of a therapeutic agent include antibiotics, steroids, anti-inflammatories, antivirals, antifungals, pain reducers, healing agents, combinations thereof, and the like. Exemplary, non-limiting antibiotics include neomycin, bacitracin, polymyxin B, mupirocin, retapamulin, clindamycin, erythromycin, silver sulfadiazine, fusidic acid, dapsone, combinations thereof, and the like, though other antibiotics are contemplated and possible. Exemplary, non-limiting steroids include clobetasol propionate, betamethasone dipropionate, mometasone furoate, triamcinolone acetonide, hydrocortisone butyrate, hydrocortisone, desonide, fluocinolone acetonide, combinations thereof, and the like, though other steroids are contemplated and possible. Exemplary, non-limiting anti-inflammatories include diclofenac, ketoprofen, piroxicam, ibuprofen, naproxen, sulindac, combinations thereof, and the like, though other anti-inflammatories are contemplated and possible. Exemplary, non-limiting antivirals include acyclovir, docosanol, penciclovir, falacyclovir, famciclovir, idoxuridine, combinations thereof, and the like, though other antivirals are contemplated and possible. Exemplary, non-limiting antifungals include clotrimazole, miconazole, terbinafine, ketoconazole, tolnafate, econazole, butenafine, ciclopirox, naftifine, oxiconazole, sulconazole, undecylenic acid combinations thereof, and the like, though other antifungals are contemplated and possible. Exemplary, non-limiting pain reduces include capsaicin, menthol, methyl salicylate, lidocaine, diclofenac, pramoxine, trolamine salicylate, arnica, benzocaine, prilocaine, combinations thereof, and the like, though other pain reducers are contemplated and possible. Exemplary, non-limiting healing agents include salicylic acid, benzoyl peroxide, tea tree oil, apple cider vinegar, aloe vera, vitamin E, witch hazel, castor oil, combinations thereof, and the like, though other healing agents are contemplated and possible.

Optionally, the skin treatment apparatus 100 may include an absorbent material disposed in the treatment space 103. In some embodiments, the absorbent material ensures the treatment media remains in the treatment space 103. In some embodiments, the absorbent material is a sponge. In some embodiments, the absorbent material may be dehydrated so as to provide a dry sponge, which may be activated with an aqueous solution before treatment, as described in greater detail herein

In some embodiments, the skin treatment apparatus 100 includes a heating element 108. In some embodiments, the heating element 108 is disposed in the treatment head 102. In some embodiments, the heating element is disposed in a housing 120. Optionally, the housing 120 is coupled to the treatment head 102. In some embodiments, the housing 120 encloses one or more additional components, such as the heating element 108 described in greater detail herein.

In some embodiments, the skin treatment apparatus 100 is powered by a power source. The power source may include a power cable electrically connecting the power source to an external power source (e.g., generator, battery, outlet, etc.). In some embodiments, the power source may include a battery, such as a lithium ion battery, optionally housed within the housing.

In some embodiments, the housing 120 may include one or more connectivity ports for interfacing with other components of the skin treatment apparatus 100, such as power inputs, data transfer ports (USB, Ethernet, etc.), and signal connectors. In some embodiments, the housing 120 encloses a controller 150, which may be used to control operation of the skin treatment apparatus 100 described in greater detail herein. For example, the controller 150 may be configured to control output of the heating element 108, initiation of a treatment protocol, and other similar functions, as will be described in additional detail herein. As such, a controller may be electrically, fluidicially, or otherwise connected to a heating element 108 such that the heating element may produce heat at a desired output for a desired duration as controlled by the controller 150.

In some embodiments, the heating element 108 is disposed in the housing 120. Any suitable heating element 108 is contemplated and possible.

In some embodiments, the heating element 108 is a resistive heating element. The resistive heating element may be provided as a wire, foil, and/or thin film. Optionally, the resistive heating element may be configured as a “two-wire” heating element, such that if functions as the heating element 108, and a temperature sensor, described in greater detail herein. It will be appreciated that a resistive heating element heats up as current passes through and the amount of heat generated is related to the resistance of the element. Heat is then transferred from the heating element 108 to the treatment media. A resistive heating element may be made from any suitable material, including, but not limited to, nickel-chromium alloys, iron-chromium-aluminum alloys, positive temperature coefficient ceramics, and the like.

In some embodiments, the heating element 108 is a conduction heating element that directly transfers heat to the treatment media through physical contact. For example, in some embodiments, a conductive heating element may be made from any electrically conductive material, such as nickel, copper, steel, metal alloys, or ceramics, that transform electrical energy, optionally from an energy generator or power source, described in greater detail herein, into heat.

In some embodiments, the heating element 108 is an induction heating element that uses electromagnetic induction to generate heat in the treatment media. For example, and without being bound by theory, an induction heating element may generate an alternating magnetic field when driven by a high-frequency current, heating up the induction heating element and transferring the heat to the treatment media. It will be appreciated that an induction heating element may enable heating of the treatment media without electrical connections.

In some embodiments, the heating element 108 is a radiant heating element that uses infrared radiation to generate heat in the treatment media. For example, in some embodiments, infrared energy passes through the treatment media and converts to heat.

Optionally, the heating element 108 is configured to transfer thermal energy to heat the treatment media to a predetermined temperature. In some embodiments, the predetermined temperature is from about 40° C. to about 100° C. or any value or range therebetween, including but not limited to about 40° C., 45° C., 50° C., 55° C., 60° C., 65° C., 70° C., 75° C., 80° C., 85° C., 90° C., 95° C., and 100° C. including any range or subrange having endpoints defined by any two of the aforementioned values.

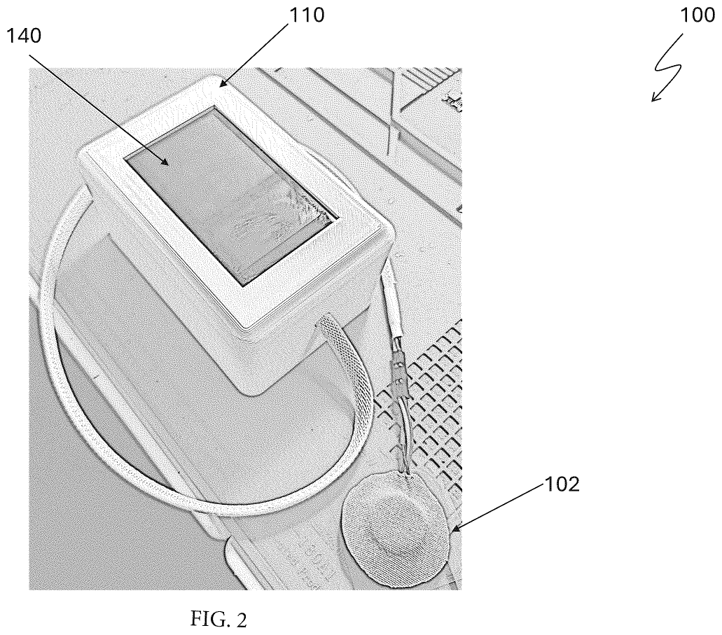

Turning now to FIG. 2, in some embodiments, the skin treatment apparatus 100 comprises an energy generator 110. Optionally, the energy generator 110 is coupled to the treatment head 102. In some embodiments, the energy generator 110 is disposed within the housing 120, described in greater detail herein. In some embodiments, such as shown in FIG. 2, the energy generator 110 is external to the treatment head 102. Any suitable energy generator 110 that is capable of transmitting energy to heat the treatment media, described in greater detail herein, is contemplated and possible. Exemplary energy generators 110 include, but are not limited to, microwave generators, laser generators, infrared generators, ultrasound generators, electrical current generators, electrochemical energy generators, mechanical energy generators, and the like. In some embodiments, the energy generator 110 is a microwave generator. In some embodiments, the energy generator 110 is an infrared generator. Optionally, the infrared generator may be a laser infrared generator.

In some embodiments, the inlet lumen transports the treatment media from the fluid reservoir to the treatment space 103 during treatment of the lesion 15. In some embodiments, the outlet lumen withdraws the treatment media from the treatment space 103. In some embodiments, the outlet lumen is coupled to a fluid waste container (not shown) which may be a vessel housing the used treatment media once it has been removed from the treatment space 103. In other embodiments, described in greater detail herein, the fluid media is withdrawn from the treatment space 103 and returned to the fluid reservoir.

In some embodiments, such as shown in FIG. 2, the skin treatment apparatus 100 may include a control panel, optionally an integrated control panel, with buttons, switches, or a touchscreen interface, allowing users to interact with the skin treatment apparatus 100. For example, and described in greater detail herein, the skin treatment apparatus 100 may include a display interface 140 through which an operator monitors and controls the skin treatment apparatus 100. In some embodiments, the display interface 140 may include indicators or alarms, such as LED indicators, audible alarms, and/or visual signals to provide real-time feedback on the status of the skin treatment apparatus 100, such as power levels, operational status, treatment parameters, or error conditions. In some embodiments, the display interface 140 may be an external device, described in greater detail herein.

The external device may include any device capable of allowing a user to interact with the skin treatment apparatus 100. In embodiments, the external device includes mobile phones, smartphones, personal digital assistants, dedicated mobile media players, mobile personal computers, laptop computers, and/or any other mobile devices capable of being communicatively coupled with the controller 150. In embodiments, the external device may include communication chips, antennas, or the like to allow the telecommunications module to communicate with others via, for example, a cellular network, WiFi, or the like.

In some embodiments, the display interface 140 includes a display, such as a graphical user interface, and/or one or more user interface controls. The display may be, for example and without limitation, any liquid crystal display (LCD), light emitting diode (LED) display, electronic ink (e-ink) display, or the like that can display information to the user. In some embodiments, the display is configured as an interactive display that can receive user input(s) (e.g., a touch screen display or the like). The user interface controls may include hardware components that receive inputs from a user and transmit signals corresponding to the inputs, such as a keyboard, a mouse, a joystick, a touch screen, a remote control, a pointing device, a video input device, an audio input device, and/or the like.

In some embodiments, the display interface 140 is configured to display one or more treatment parameters. In some embodiments, the display interface 140 is configured to display one or more of treatment protocol selected, treatment duration remaining, treatment duration elapsed, temperature of the treatment media, temperature of the treatment space 103, pressure in the treatment space 103, and/or error messages. In some embodiments, described in greater detail herein, the display interface 140 is configured to allow an operator to navigate through different settings and modes, select desired functions, and/or adjust treatment parameters. In some embodiments, the display interface 140 is configured to display various data related to the treatment protocol at the conclusion of the treatment. Exemplary data includes minimum and/or maximum temperatures, average temperature for the duration of the treatment protocol, minimum and/or maximum pressures, average pressure for the duration of the treatment protocol, operating status of the various components, and the like.

In some embodiments, the skin treatment apparatus 100 includes a user input configured for one or more of activating the heating element 108, initiating a treatment protocol, pausing a treatment protocol, deactivating the heating element 108, terminating a treatment protocol, and/or changing a display interface, described in greater detail herein. In some embodiments, the user input is a selectable option on the user interface 140.

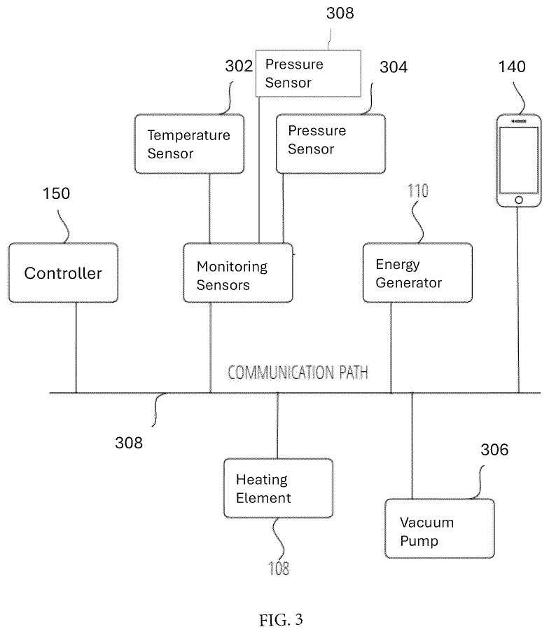

Turning now to FIG. 3, a schematic of a system 300 for operating the skin treatment apparatus is depicted. In some embodiments, the skin treatment apparatus 100 includes a temperature sensor 302 to monitor and/or control the temperature of the treatment media, described in greater detail herein. The temperature sensor 302 may be any sensor capable of collecting data related to the temperature of the environment it is positioned within and producing a signal indicative of the temperature. Optionally, the temperature sensor 302 may contact the heating element 108 and/or the treatment media and/or the treatment space 103. Illustrative, non-limiting examples of temperature sensors 302 include thermocouples, resistance temperature detectors, thermistors, semiconductor temperature sensors, infrared temperature sensors, bimetallic strips, and the like.

In some embodiments, the skin treatment apparatus 100 comprises one or more impedance sensors (308). Optionally, the impedance sensor 308 is disposed in the treatment space 103 to monitor the electrical impedance before, after, or during treatment of the lesion 15. The impedance sensor 308 optionally is an electrode pair or a multiplexed electrode array (e.g., 2-, 3-, or 4-electrode configurations). In some embodiments, the impedance sensor 308 is a three-electrode configuration. In some embodiments, the impedance sensor 308 is configured to output a low-amplitude, multi-frequency AC stimulus (e.g., 1 kHz-5 MHz) while simultaneously acquiring voltage and current to compute complex impedance. In some embodiments,

Optionally, the skin treatment apparatus 100 comprises one or more pressure sensors 304 disposed in the treatment space 103 to monitor the pressure during treatment of the lesion 15. Exemplary pressure sensors include, but are not limited to, strain gauge pressure sensors, capacitive pressure sensors, piezoelectric pressure sensors, optical pressure sensors, resistive pressure sensors, electromagnetic pressure sensors, silicon pressure sensors, and the like, though any suitable pressure sensor is contemplated and possible.

For example, and without being bound by theory, in some embodiments, the skin treatment apparatus 100 includes a vacuum pump 306. In some embodiments, the vacuum pump 306 is configured to create a pressure differential between the treatment space 103 and the external environment. In some embodiments, the vacuum pump 306 is fluidically coupled to the treatment head 102 via one or more conduits. In some embodiments, the vacuum pump 306 is configured to remove air from the treatment space 103, thereby causing the pressure of the external environment to press the treatment head 102 against the treatment surface 10. In some embodiments, the vacuum pump 306 is used to exert negative pressure against the treatment surface 10.

In some embodiments, the treatment surface 10 is pulled into the treatment space 103 during operation of the vacuum pump 306. Optionally, the vacuum pump 306 exerts enough negative pressure to separate the epidermis from the dermis. It will be appreciated that the separation of the epidermis from the dermis may stimulate various biological responses, such as increased blood flow to the treatment surface 10, which may promote healing or the delivery of treatments to the lesion and to deeper skin layers. In some embodiments, the separation of the epidermis from the dermis may trigger the production of new skin cells.

As noted briefly above, the skin treatment apparatus may include a controller 150. In some embodiments, the controller 150 is configured to control and operate the skin treatment apparatus 100. In some embodiments the controller 150 is communicatively coupled to one or more components of the skin treatment apparatus 100. In some embodiments, the various components of the skin treatment apparatus 100, such as the heating element 108, the energy generator 110, the user interface 140, the temperature sensor(s) 302 and/or pressure sensors 304 are communicatively coupled via a communication path 308. As used herein, the term “communicatively coupled” means that coupled components are capable of exchanging data signals with one another such as, for example, electrical signals via conductive medium, electromagnetic signals via air, optical signals via optical waveguides, and the like. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium.

In some embodiments, the communication path includes a conductive material that permits the transmission of electrical data signals to processors, memories, controllers, or sensors throughout the skin treatment apparatus 100. The communication path may be formed from any medium that is capable of transmitting a signal such as, for example, conductive wires, conductive traces, optical waveguides, or the like. In some embodiments, the communication path facilitates the transmission of wireless signals, such as WiFi, Bluetooth, and the like. Moreover, the communication path may be formed from a combination of mediums capable of transmitting signals. For example, the communication path may comprise a combination of conductive traces, conductive wires, connectors, and buses that cooperate to permit the transmission of electrical data signals to components such as processors, memories, sensors, input devices, output devices, and communication devices. Accordingly, the communication path may comprise a vehicle bus, such as for example a LIN bus, a CAN bus, a VAN bus, and the like.

In some embodiments, the controller 150 is communicatively coupled to the heating element 108, the energy generator 110, the vacuum pump 306, the display interface 140, the temperature sensor(s) 302, the pressure sensor(s) 304, and/or the impedance sensor(s) 308. In embodiments, the controller 150 is communicatively coupled to element 108, the energy generator 110, the vacuum pump 306, the display interface 140, the temperature sensor(s) 302, the pressure sensor(s) 304, and/or the impedance sensor(s) 308 by the communication path. In embodiments, the controller 150 is configured to control various operations of the system 300.

In some embodiments, the controller 150 is implemented using integrated and/or discrete hardware elements, software elements, and/or a combination thereof. Examples of integrated hardware elements include, but are not limited to, processors, microprocessors, microcontrollers, integrated circuits, application specific integrated circuits (ASIC), programmable logic devices (PLD), digital signal processors (DSP), field programmable gate arrays (FPGA), logic gates, registers, semiconductor devices, chips, microchips, chip sets, microcontrollers, system-on-chip (SoC), and/or system-in-package (SIP). Examples of discrete hardware elements include, but are not limited to, circuits and/or circuit elements such as logic gates, field effect transistors, bipolar transistors, resistors, capacitors, inductors, and/or relays. In some embodiments, the controller 150 includes a hybrid circuit comprising discrete and integrated circuit elements or components. By executing instruction code stored in a memory module, the controller 150 may control various components of the skin treatment apparatus 100, such as the heating element 108, the energy generator 110, the vacuum pump 306, the display interface 140, the temperature sensor(s) 302, the pressure sensor(s) 304, and/or the impedance sensor(s) 308, as well as control the various parameters of the treatment protocols, such as predetermined temperature, pressure levels, and/or treatment duration, for example.

In some embodiments, the controller 150 is configured to transmit data to the display interface 140. As noted briefly above, in some embodiments, the display interface 140 may be disposed on the skin treatment apparatus 100 or may be an external device. In embodiments, the data is transmitted using a communications module. In embodiments, the data is the signal output from the temperature sensor(s) 302 the pressure sensor(s) 304, and/or the impedance sensor(s) 308. In embodiments, the data is analyzed and/or processed by the controller 150 before transmission.

Optionally, the controller 150 is a programmable device that receives the signals from the temperature sensor(s) 302, the pressure sensor(s) 304, and/or the impedance sensor(s) 308 s, processes the information according to instructions in the memory module, and provides an output. For example, and not being bound by theory, if a temperature sensor detects a temperature of the treatment media that is below the predetermined temperature, the controller may modulate the output of the energy generator 110 to increase the energy to the heating element 108, thereby increasing the temperature of the treatment media. If In some embodiments, if the pressure sensor 304 sends a signal and the controller 150 determines the pressure of the treatment space 103 falls outside the set parameters, the controller 150 may reactivate the vacuum pump 306 to modulate the pressure in the treatment space 103.

In some embodiments, the impedance sensor 308 is configured to send output information to the controller 150. In some embodiments, the controller 150 executes a bioimpedance analysis in which the frequency response is fit to an equivalent electrical model (e.g., a two-or three-dispersion Cole/Cole-Cole model) that parameterizes stratum corneum resistance, viable epidermis/dermis extracellular resistance, intracellular/vascular contributions, and/or interfacial or membrane capacitances, yielding frequency-dependent conductance and aggregated skin conductance. For example, and without being bound by theory, by analyzing the shift of dispersion corner frequencies and the ratio of high-to low-frequency conductance, the controller 150 estimates local skin and soft-tissue thickness, for example by correlating the depth of current penetration (which scales with frequency and tissue conductivity) to stored calibration curves derived from tissue-equivalent phantoms and/or ultrasound-validated patient datasets.

In some embodiments, the controller 150 may further compensate estimates for contact pressure (from pressure sensor (304)), temperature (from temperature sensor (302)), and electrode geometry via per-device calibration constants, and rejects motion artifacts via coherence/phase-stability checks. Optionally, using the derived parameters, the system 300 may select or adapt a treatment protocol. For example, and without being bound by theory, in some embodiments, the system 300 selects a target treatment to achieve a desired thermal dose at a depth proportion to the thickness of the skin. In some embodiments, the system 300 selects a dwell time and duty cycle to maintain the therapeutic effect while limiting peak epidermal temperature when the conductance indicates specific conditions, such as high hydration or low barrier function. In some embodiments, the system 300, selects a pulse sequencing treatment protocol (e.g., continuous vs. intermittent heating) to allow conductive/convective recovery in thin or highly conductive ski. In some embodiments, the system 300 triggers safety cutoffs that terminate the treatment protocol, such as when real-time impedance tracking shows barrier compromise (rapid drop in stratum corneum resistance) or edema (phase shift drift).

Referring again jointly to FIGS. 1-3, in some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media.

In some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media. In some embodiments, the treatment media is a fluid or a gel.

In some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media. In some embodiments, the skin treatment apparatus 100 includes a temperature sensor 302. Optionally, the temperature sensor 302 is configured to contact the heating element 108 or the treatment media.

In some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media. In some embodiments, the skin treatment apparatus 100 includes a temperature sensor 302. Optionally, the temperature sensor 302 is configured to contact the heating element 108 or the treatment media. In some embodiments, the skin treatment apparatus includes a controller 150. Optionally, the controller 150 is communicatively coupled to the heating element 108. In some embodiments, the controller 150 is communicatively coupled to the temperature sensor 302. In some embodiments, the controller 150 is configured to receive a signal from the temperature sensor 302. Optionally, the controller 150 is configured to modulate the output of the heating element 108 based on the signals from the temperature sensor 302.

In some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media. In some embodiments, the skin treatment apparatus 100 includes an impedance sensor 308.

In some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media. In some embodiments, the skin treatment apparatus 100 includes an impedance sensor 308. In some embodiments, the skin treatment apparatus 100 includes a controller 150. Optionally, the controller 150 is communicatively coupled to the heating element 108. In some embodiments, the controller 150 is communicatively coupled to the impedance sensor 308. In some embodiments, the controller 150 is configured to receive a signal from the impedance sensor 308.

In some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media. In some embodiments, the skin treatment apparatus 100 includes a pressure sensor 304.

In some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media. In some embodiments, the skin treatment apparatus 100 includes a pressure sensor 304. In some embodiments, the skin treatment apparatus 100 includes a controller 150. Optionally, the controller 150 is communicatively coupled to the heating element 108. In some embodiments, the controller 150 is communicatively coupled to the pressure sensor 304. In some embodiments, the controller 150 is configured to receive a signal from the pressure sensor 308.

In some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media. In some embodiments, the skin treatment apparatus 100 includes a temperature sensor 302 and a pressure sensor 304. In some embodiments, the skin treatment apparatus 100 includes a temperature sensor 302 and an impendence sensor 308. In some embodiments, the skin treatment apparatus 100 includes a pressure sensor 304 and an impedance sensor 308. In some embodiments, the skin treatment apparatus 100 includes a temperature sensor 302, a pressure sensor 304, and an impendence sensor 308.

In some embodiments, the skin treatment apparatus 100 includes a treatment head 102 defining a treatment space 103, the treatment head comprising a body 104 forming said treatment space 103, wherein said body 104 is further configured to contact the treatment space 103 to a treatment surface 10 comprising a lesion 15. In some embodiments, the skin treatment apparatus 100 includes a treatment media disposed within the treatment space 103. In some embodiments, the skin treatment apparatus 100 includes a heating element 108 configured to heat the treatment media. In some embodiments, the skin treatment apparatus 100 includes a controller 150. Optionally, the controller 150 is communicatively coupled to the heating element 108. In some embodiments, the skin treatment apparatus 100 includes a temperature sensor 302 and a pressure sensor 304. In some embodiments, the controller 150 is communicatively coupled to the temperature sensor 302 and the pressure sensor 304. In some embodiments, the controller 150 is configured to receive a signal from the temperature sensor 302 and the pressure sensor 304. In some embodiments, the skin treatment apparatus 100 includes a temperature sensor 302 and an impendence sensor 308. In some embodiments, the controller 150 is communicatively coupled to the temperature sensor 302 and the impedance sensor 308. In some embodiments, the controller 150 is configured to receive a signal from the temperature sensor 302 and the impedance sensor 308. In some embodiments, the skin treatment apparatus 100 includes a pressure sensor 304 and an impedance sensor 308. In some embodiments, the controller 150 is communicatively coupled to the pressure sensor 304 and the impedance sensor 308. In some embodiments, the controller 150 is configured to receive a signal from the pressure sensor 304 and the impedance sensor 308. In some embodiments, the skin treatment apparatus 100 includes a temperature sensor 302, a pressure sensor 304, and an impendence sensor 308.

In some embodiments, the controller 150 is communicatively coupled to the temperature sensor 302, the pressure sensor 304, and the impedance sensor 308. In some embodiments, the controller 150 is configured to receive a signal from the temperature sensor 302, the pressure sensor 304, and the impedance sensor 308.

Also provided herein are methods of treating a skin condition in a subject, optionally using a device as described briefly herein. In some embodiments, the present disclosure relates to methods of treating a lesion on the skin. In some embodiments, the methods of treating the skin condition and/or the lesion use the skin treatment apparatus 100 or as otherwise provided herein.

In an embodiment, the present disclosure relates to a method of treating a lesion using the skin treatment apparatus, including sealing the treatment head to a treatment surface comprising the lesion; initiating a treatment protocol, the treatment protocol having a treatment duration; and contacting the lesion with the heated treatment media for the treatment duration.

In some embodiments, the lesion is selected from warts, acrochordons, skin cancer, corns, calluses, actinic keratosis, molluscum contgaiosum, dermatosis papulosa nigra, solar lentigines, cutaneous horns, lichen planus, keloids, pyogenic granulomas, or granuloma annulare, optionally, warts.

In some embodiments, the treatment duration is from about 10 seconds to about 60 seconds, or about 1 minute to about 60 minutes, or about 5 minutes to about 60 minutes, or from from about 5 minutes to about 30 minutes, including 10 seconds, 15 seconds, 30 seconds, 45 seconds, 60 seconds, 90 seconds, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, and 60 minutes, including any subrange defined by any two of the aforementioned endpoints.

In some embodiments, the treatment protocol further includes heating the fluid to a predetermined temperature, wherein the predetermined temperature is from about 40° C. to about 100° C., or any value or range therebetween, including but not limited to about 40° C., 45° C., 50° C., 55° C., 60° C., 65° C., 70° C., 75° C., 80° C., 85° C., 90° C., 95° C., and 100° C., including any subrange defined by any two of the aforementioned values.

Referring jointly to FIGS. 1-3, in some embodiments, an operator selects a treatment protocol using the display interface 140 of the skin treatment apparatus 100. In some embodiments, the operator may select a treatment head 102 and couple the treatment head to the housing 120.

In some embodiments, the treatment head 102 is sealed to the treatment surface 10 including the lesion 15. In some embodiments, the treatment head 102 is selected based on the size of the lesion 15. Optionally, the treatment head 102 is sealed to the treatment surface 10 with a lip 106 of the treatment head 102. In some embodiments, the operator may select a lip 106 and attach the lip 106 to the body 104 of the treatment head 102.

In some embodiments, the operator activates the vacuum pump 306 to seal the treatment head 102 to the treatment surface 10. In some embodiments, the vacuum pump 306 sucks the epidermis into the treatment space 103, thereby separating the epidermis from the dermis. In some embodiments, the controller 150 activates the vacuum pump 306, to seal the treatment head 102 to the treatment surface 10.

In some embodiments, the operator initiates the treatment protocol. Optionally, the heating element 108 heats the treatment media to a predetermined temperature. In some embodiments, the predetermined temperature is from about 40° C., 45° C., 50° C., 55° C., 60° C., 65° C., 70° C., 75° C., 80° C., 85° C., 90° C., 95° C., and 100° C., including any range or subrange having endpoints defined by any two of the aforementioned values. In some embodiments, the skin treatment apparatus 100 includes a temperature sensor 302 to monitor and/or control the temperature of the treatment media. Optionally, once the temperature sensor 302 detects the treatment media has reached the predetermined temperature, the controller 150 modifies an output of energy from the heating element 108 to maintain the predetermined temperature.

In some embodiments, the lesion 15 is contacted with the heated treatment mediafor the treatment duration. In some embodiments, the operator may select and/or adjust the treatment duration on the display interface 140. In some embodiments, the treatment duration is included in the treatment protocol. Optionally, the treatments duration is from about 10 seconds to about 60 minutes, including about 10 seconds, 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, and 60 minutes, including any range or subrange having endpoints defined by any two of the aforementioned values. In some embodiments, the treatment duration is from about 10 seconds to about 60 seconds, or about 1 minute to about 60 minutes, or about 5 minutes to about 60 minutes, or from about 5 minutes to about 30 minutes.

In some embodiments, the lesion is pared after the treatment. In some embodiments, a topical medication is applied to the lesion.

In some embodiments, the methods of treating the skin condition and/or lesion include treatment protocols having a plurality of cycles. In some embodiments, each cycle includes heating the treatment media to a first predetermined temperature and maintaining the treatment media at the first predetermined temperature for a first treatment time. Optionally, the heating elements is deactivated for a second treatment time.

In some embodiments, the first predetermined temperature is from about 40° C., 45° C., 50° C., 55° C., 60° C., 65° C., 70° C., 75° C., 80° C., 85° C., 90° C., 95° C., and 100° C., including any range or subrange having endpoints defined by any two of the aforementioned values. In some embodiments, the first treatment time is from about 10 seconds to about 60 minutes, including about 10 seconds, 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, and 60 minutes, including any range or subrange having endpoints defined by any two of the aforementioned values.

In some embodiments, deactivating the heating element 108 allows the temperature in the treatment space to return to approximately 37° C. In some embodiments, the second treatment time from about 10 seconds to about 60 minutes, including about 10 seconds, 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, and 60 minutes, including any range or subrange having endpoints defined by any two of the aforementioned values.

In some embodiments, the methods of treating the skin condition and/or lesion include multiple applications of the treatment protocol. In some embodiments, an operator may perform the treatment protocol, multiple times per day (twice daily, three times daily, four times daily, etc.), daily, bi-weekly, weekly, bimonthly, monthly, etc. In some embodiments, the treatment protocol is performed daily. Optionally, the treatment protocol is performed daily for a week. Optionally, the treatment protocol is performed daily for two weeks. Optionally, the treatment protocol is performed daily for three weeks. Optionally, the treatment protocol is performed daily for four weeks. Optionally, the treatment protocol is performed daily for five weeks. Optionally, the treatment protocol is performed daily for six weeks. In some embodiments, the treatment protocol is performed daily until the lesion size reduces. In some embodiments, the treatment protocol is performed daily until the wart size is reduced. Optionally, the treatment protocol is performed daily until the lesion resolves. Optionally, the treatment protocol is performed daily until the wart resolves. Optionally, the treatment protocol is performed daily for three weeks.

In some embodiments, the treatment protocol is performed weekly for four weeks. Optionally, the treatment protocol is performed weekly for five weeks. Optionally, the treatment protocol is performed weekly for six weeks. In some embodiments, the treatment protocol is performed weekly until the lesion size reduces. In some embodiments, the treatment protocol is performed weekly until the wart size reduces. Optionally, the treatment protocol is performed weekly until the lesion resolves. Optionally, the treatment protocol is performed weekly until the wart resolves.

It will be appreciated that skin treatment apparatus 100 of the present disclosure creates a hydrated, hyperthermic environment around the lesion for the treatment duration. This environment aids in treating the lesion by hydrating the corneal layer, expanding compressed tissue, separating living and dead cells, conducting heat into the dermis.

### EXAMPLES

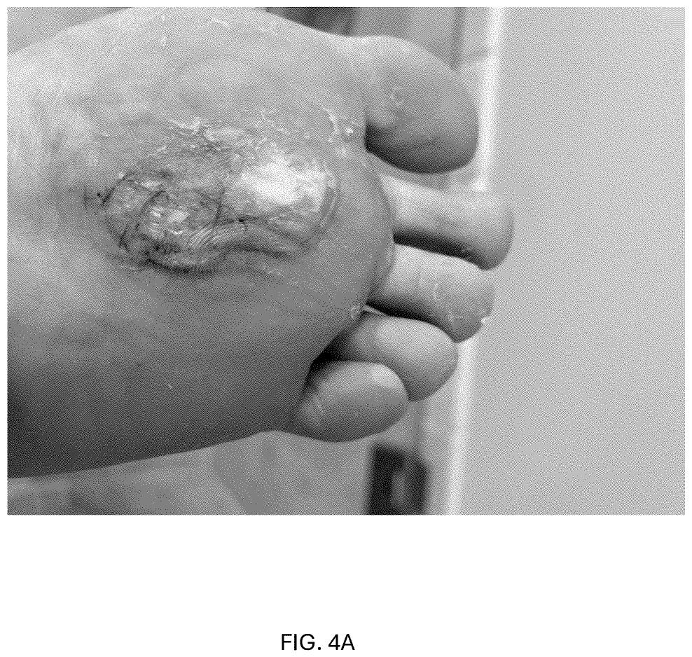

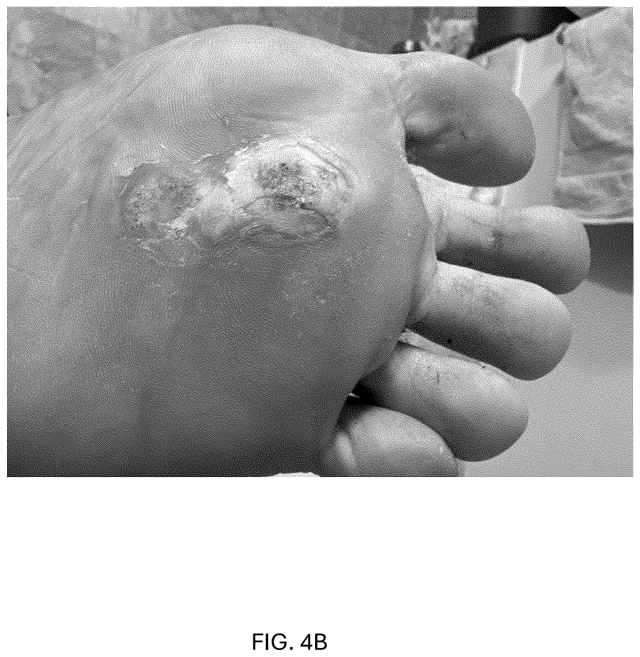

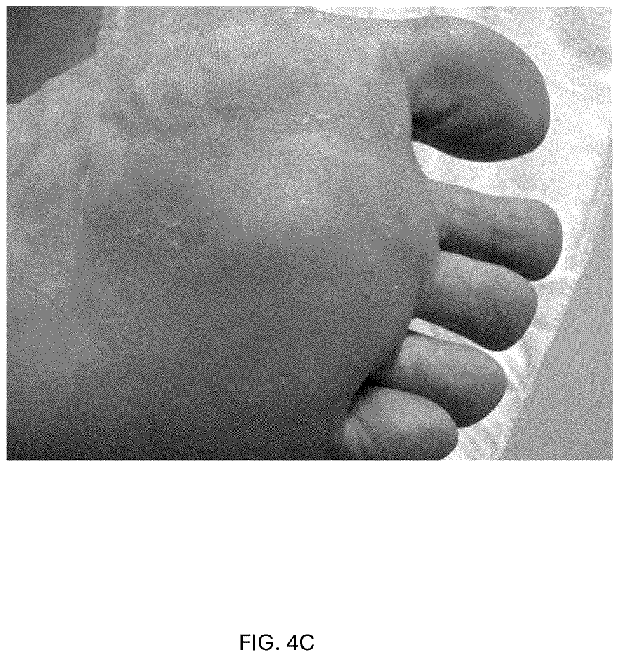

A 37-year-old male presented with one plantar wart, about 1.5 cm in diameter, shown in FIG. 4A. He stated that the wart first appeared 3 years prior and had not spontaneously resolved as hoped. The wart was pared and cryotherapy was applied 3-5 times in five second bursts. Cryotherapy was repeated every four weeks for four sessions. No improvement was noted. Additional lesions were observed below the primary lesion by the conclusion of this treatment.

Various other treatments were prescribed in conjunction with subsequent cryotherapy. After 7 cryotherapy sessions, efudex application, cantharidin treatment, imiquimod treatment, and 4 bleomycin intralesional injections in 13 months, no significant improvement was apparent in the lesion, shown in FIG. 4B. Additionally, the patient reported significant pain and difficulty walking.

The patient underwent hydro-hyperthermia treatment. Each treatment involved 20 minutes at 52° C. to 56° C. The treatment was repeated 4-5 times per week for 6 weeks. The lesion was pared and efudex was applied to the lesion after each treatment. It was noted paring the lesion was easier following hydro-hypothermic treatment because the active lesion tissue would separate from the dead hyperkeratotic skin layers.

As shown in FIG. 4C, at the conclusion of the hydro-hyperthermic treatment, the lesions were resolved. Notably, no sign of active infection was observed, no capillaries were present in the epithelium, and the deep fibrous swelling around the primary lesion was gone.

What is claimed is:

1. A skin treatment apparatus comprising:
a treatment head defining a treatment space, the treatment head comprising a body forming said treatment space, wherein said body is further configured to contact the treatment space to a treatment surface comprising a lesion;
a treatment media disposed within the treatment space; and
a heating element configured to heat the treatment media.

2. The skin treatment apparatus of claim 1, wherein the heating element is configured to heat the treatment media to a predetermined temperature, wherein the predetermined temperature is from about 40° C. to about 100° C.

3. The skin treatment apparatus of claim 1, further comprising a temperature sensor contacting the heating element and/or the treatment media.

4. The skin treatment apparatus of claim 1, further comprising a display interface configured to display one or more parameters selected from treatment protocol, treatment duration remaining, treatment duration elapsed, temperature of the treatment media, or error messages.

5. The skin treatment apparatus of claim 1, wherein the skin treatment apparatus is configured to send or receive data about a treatment to an external device.

6. The skin treatment apparatus of claim 1, wherein the treatment media is a fluid or a gel.

7. The skin treatment apparatus of claim 1, wherein the treatment media comprises one or more therapeutic agents.

8. The skin treatment apparatus of claim 1, wherein the treatment head seals to the treatment surface adjacent to the lesion via adhesion.

9. The skin treatment apparatus of claim 1, wherein the treatment head seals to the treatment surface adjacent to the lesion via suction.

10. The skin treatment apparatus of claim 1, further comprising a controller communicatively coupled to the heating element, the controller configured to activate the heating element during a treatment protocol.

11. The skin treatment apparatus of claim 1, further comprising a vacuum pump, wherein the vacuum pump is configured to remove air from the treatment space.

12. The skin treatment apparatus of claim 1, further comprising an impendence sensor.

13. A method of treating a lesion using the apparatus of claim 1, the method comprising:
sealing the treatment head to a treatment surface comprising the lesion;
initiating a treatment protocol, the treatment protocol having a treatment duration;
heating the treatment media to a predetermined temperature; and
contacting the lesion with the heated treatment media for the treatment duration.

14. The method of claim 13, wherein the lesion is selected from warts, bacterial infections, viral infections, insect bites, acrochordons, skin cancer, corns, calluses, actinic keratosis, molluscum contgaiosum, dermatosis papulosa nigra, solar lentigines, cutaneous horns, lichen planus, keloids, pyogenic granulomas, or granuloma annulare.

15. The method of claim 13, wherein the lesion is a wart.

16. The method of claim 13, wherein the treatment duration is from about 10 seconds to about 60 seconds.

17. The method of claim 13, wherein the treatment duration is from about 1 minute to about 60 minutes.

18. The method of claim 13, wherein the predetermined temperature is from about 40° C. to about 100° C.

19. The method of claim 13, wherein sealing the treatment head to a treatment surface comprises activating a vacuum pump to remove air from the treatment space.

20. The method of claim 13, wherein the treatment protocol further comprises a plurality of cycles, wherein each cycle comprises:
heating the treatment media to a first predetermined temperature and maintaining the treatment media at the first predetermined temperature for a first treatment time;
deactivating said heating element for a second treatment time.
